(19) United States
(12) Patent Application Publication
(10) Pub. No.: US 2015/0094740 A1
(43) Pub. Date: Apr. 2, 2015

(54) Methods and Systems for Ligating a Blood Vessel

(71) Applicant: Paul J. GAGNE, (US)

(72) Inventor: Paul J. Gagne, Darien, CT (US)

(21) Appl. No.: 14/390,652

(22) PCT Filed: Mar. 14, 2013
(86) PCT No.: PCT/US13/31209

§ 371 Date: Oct. 3, 2014

Related U.S. Application Data
(60) Provisional application No. 61/620,171, filed on Apr. 4, 2012.
(60) Provisional application No. 61/644,666, filed on May 9, 2012.

(51) Int. Cl. A61B 17/12; A61B 17/06

(52) U.S. Cl. CPC A61B 17/12013; A61B 17/06166; A61B 17/06066; A61B 2017/00778
USPC 606/144

(57) ABSTRACT

Systems and methods for ligation of a blood vessel are disclosed. In some embodiments, the systems and methods include the following: a delivery device including a needle; a space creating mechanism for creating space outside near and far walls of the vessel; a suture ligator including a far wall buttress that is positioned outside the far wall of the vessel via the needle; a near wall buttress that is positioned and secured along the suture ligator outside the near wall of the vessel via the needle; and a plunger for positioning the near wall buttress downwardly at a position along the suture ligator so that the near wall buttress compresses the vessel but is not joined with the far wall buttress; and a locking mechanism for securing the near wall buttress to the suture ligator at a position proximate to the far wall buttress thereby ligating the vessel.

### CROSS REFERENCE TO RELATED APPLICATION(S)

This application claims the benefit of U.S. Provisional Application Nos. 61/620,171, filed Apr. 4, 2012, and 61/644,666, filed May 9, 2012, which are incorporated by reference as if disclosed herein in its entirety.

### BACKGROUND

Superficial varicose veins and venous insufficiency can be cosmetically unattractive and also lead to chronic leg pain, aching, swelling, skin discoloration, and venous ulcers. Many techniques exist to treat superficial varicose veins. Minimally invasive techniques with catheter based ablation has been developed to treat long/straight superficial “trunk” veins such as the greater and short saphenous vein. The anterolateral branch of the sapheno-femoral junction can also sometimes be closed in this manner. These minimally invasive catheter based techniques have largely replaced conventional vein stripping in the United States.

Unfortunately, many patients have large, painful varicose veins that are too short or too tortuous to close with catheter ablation techniques. These veins are addressed using stab phlebectomy, an invasive surgical technique, or injection sclero therapy with a liquid or foam. Foam injections have been associated with complications such as deep vein thrombosis, stroke, dry cough, headache, numbness of a part of the body, fainting, or disturbance of vision. This is felt to be due to diffusion of the foam sclerosant, an irritating chemical injected into the vein, which spreads (or flows, leaches), into other parts of the vascular system. Furthermore, the effectiveness of foam sclerotherapy for ablating the greater saphenous vein is approximately 70%, which is inferior to other techniques. If the foam could be localized to the target varicose vein, complications related to the spread of the sclerosant to other vascular beds would be alleviated. Also, better localization and concentration of the sclerosant to the target vein would lead to improved vein closure and alleviation of the symptoms of chronic venous insufficiency related to varicose veins.

There are various known techniques to ligate or interrupt a blood vessel: 1) incision and suture ligation of the vessel; 2) percutaneous chemical ablation of the vessel with glue or sclerosant; 3) percutaneous “coil” or “cuff” embolization; 4) percutaneous catheter ablation of the vessel using heat, chemical, or mechanical disruption; and 5) incision and mechanical removal, e.g., avulsion, etc., of the blood vessel.

Techniques including incision are invasive, painful, and often require a lengthy recovery period. The use of chemical ablation often includes side effects and can negatively impact surrounding or remote tissue and organs. Percutaneous techniques such as coil or cuff embolization and the use of heat, chemical, or mechanical disruption are often painful and can also include harmful side effects to a patient.

### SUMMARY

Aspects of the disclosed subject matter include systems and methods for ligating blood vessels such as veins. In some embodiments, buttresses are positioned outside the near and far walls of a blood vessel along a suture ligator such that the blood vessel is compressed between the buttresses thereby interrupting blood flow and achieving ligation of the vessel. The buttresses are positioned via a needle and typically under ultrasound guidance. The buttresses are not joined together but rather brought into close proximity with one another.

### BRIEF DESCRIPTION OF THE DRAWINGS

The drawings show embodiments of the disclosed subject matter for the purpose of illustrating the invention. However, it should be understood that the present application is not limited to the precise arrangements and instrumentalities shown in the drawings, wherein:

FIG. 1 is an enlarged schematic diagram of methods and systems according to some embodiments of the disclosed subject matter;

FIGS. 2A-2H is a schematic diagram of methods and systems according to some embodiments of the disclosed subject matter;

FIGS. 3 is an enlarged schematic diagram of a buttress according to some embodiments of the disclosed subject matter;

FIG. 4 is an enlarged schematic diagram of a buttress according to some embodiments of the disclosed subject matter;

FIGS. 5A-5H are a schematic diagram of methods and systems according to some embodiments of the disclosed subject matter;

FIGS. 6A-6C are enlarged schematic diagrams of locking mechanisms according to some embodiments of the disclosed subject matter;

FIGS. 7A-7E are enlarged schematic diagrams of methods and systems according to some embodiments of the disclosed subject matter; and

FIG. 8 is a chart of a method according to some embodiments of the disclosed subject matter.

### DETAILED DESCRIPTION

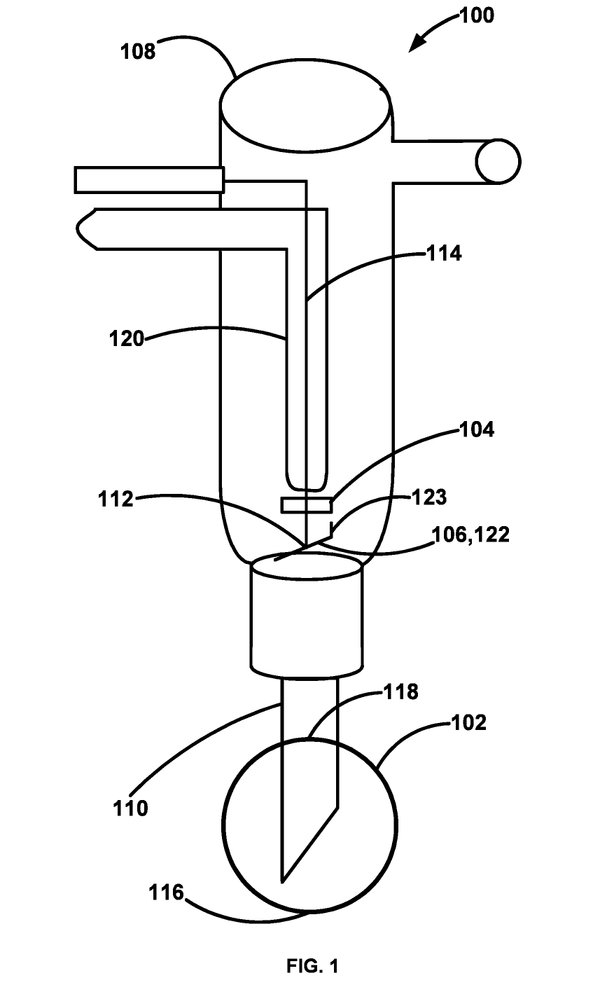

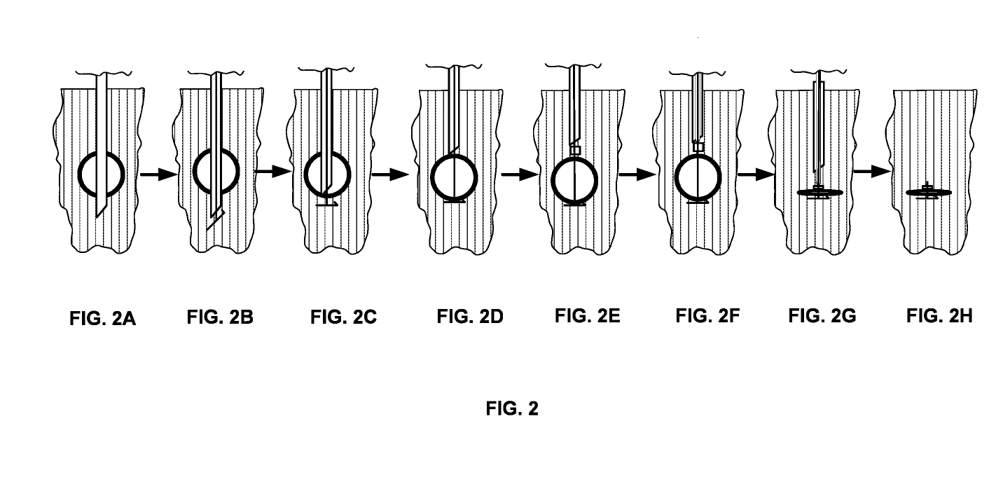

Referring now to FIGS. 1 and 2, some embodiments of the disclosed subject matter include a system 100 for ligation of a vessel 102, e.g., a vein. In some embodiments, system 100 includes near and far wall buttresses 104 and 106, respectively, that are positioned proximate one another on opposite sides of vessel 102 to compress and ligate the vessel. Although not shown, in some embodiments, placement of near and far wall buttresses 104 and 106 is facilitated with the use of a guidewire, delivery catheter, and ultrasound guidance.

Some embodiments of system 100 include a delivery device 108 having a needle 110 through which near and far wall buttresses 104 and 106 are positioned within a patient. Because needle 110 is typically placed using ultrasound guidance, the needle used is typically hyperechoic. Far wall buttress 106 is formed at or joined with an end 112 of a suture ligator 114 and is positioned outside a far wall 116 of vessel 102 via needle 110. Near wall buttress 104 is positioned and secured along suture ligator 114 outside a near wall 118 of vessel 102 via needle 110.

In some embodiments, system 100 includes a plunger 120 for positioning near wall buttress 104 downwardly against vessel 102 and securing the near wall buttress proximate to but not joined with far wall buttress 106 thereby ligating the vessel. Plunger 120 is sized to fit within needle 110.

Still referring to FIGS. 1 and 2A-2H, in some embodiments, near wall buttress 104 is formed from a disk or bar and far wall buttress 106 includes a barbed shoe 122, and both are formed from inert materials such as stainless steel. Barbed shoe 122 includes a barbed portion 123 that catches in the tissue to gain leverage to secure the shoe against far wall 116 of vessel 102. The length and width of near wall buttress 104 and far wall buttress 106 are selected so as to optimize 100% ligation of vessel 102. Of course, as one skilled in the art will appreciate, far wall buttress 106 can be formed from myriad other designs.

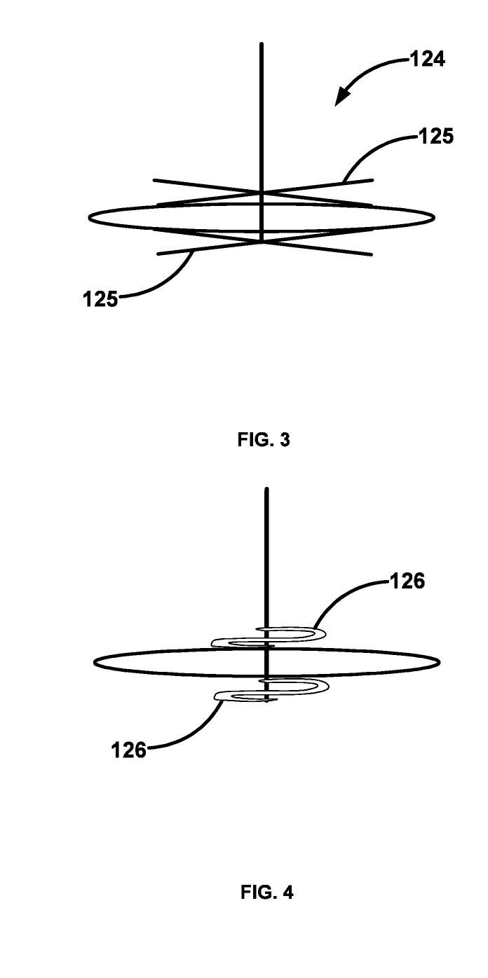

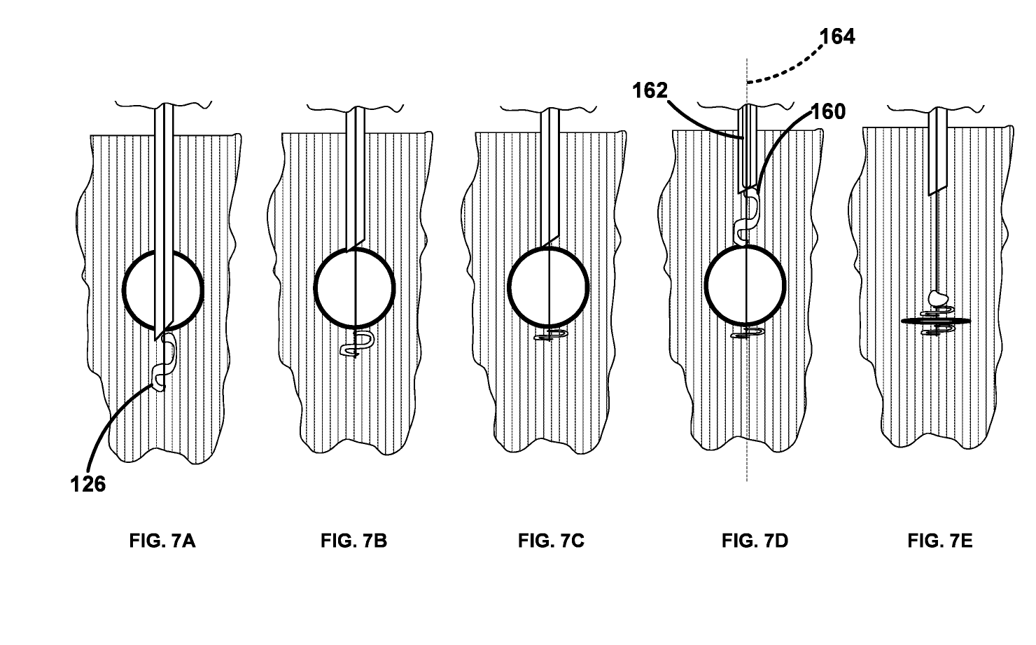

Referring now to FIG. 3 and as discussed further below, in some embodiments, far wall buttress 106 is formed from a star-shaped member 124. Member 124 can be collapsed like an umbrella to remove from the vein/patient if desired after temporary vessel ligation has been achieved. Referring now to FIG. 4 and as discussed further below with respect to FIG. 7A-7E, in some embodiments, far wall buttress 106 is formed from a collapsible accordion portion 126.

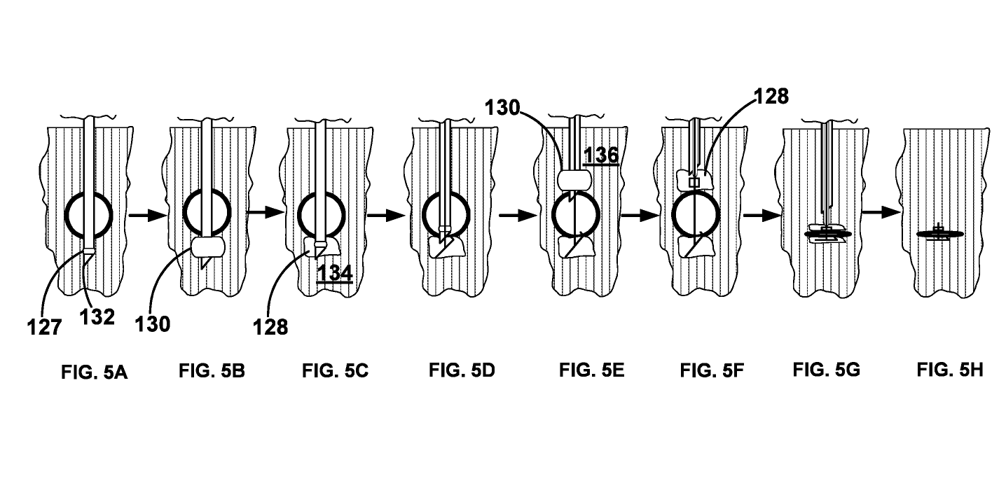

Referring now to FIGS. 5A-5H, in some embodiments, system 100 includes a space creating mechanism 127 for creating spaces 128 outside near wall 118 of vessel 102 and outside a far wall 116 of the vessel. Space creating mechanism 127 helps achieve reproducible and accurate approximation of far wall buttress 106 of suture ligator 114 to far wall 116 of vessel 102. In some embodiments, space creating mechanism 127 includes a balloon 130 that is placed on needle 110 adjacent a tip 132 of the needle. In embodiments where a catheter is used (not shown), balloon 130 is adjacent an end or tip of the catheter. Once needle 110 has penetrated through the near and far walls 118, 116 of vessel 102, which is typically performed under ultrasound guidance, balloon 130 is inflated to create space 128 in a soft connective tissue 134 deep to the vessel. This allows far wall buttress 106, when deployed, to be less likely to become entrapped in tissue around vessel 102 and not seat against the vessel properly. Once space 128 has been created, balloon 130 is deflated and then far wall buttress 106 of suture ligator 114 is deployed and needle 110 withdrawn to an area 136 adjacent near wall 118 of vessel 102. Balloon 130 is then re-inflated to create space 128 in area 136 and near wall buttress 104 is deployed in the area so as to engage vessel 102 and insure unhindered cinching of suture ligator 114 and ligation of the vessel.

In some embodiments, instead of balloon 130, space creating mechanism 127 includes a solution (not shown), e.g., saline or tumescent anesthesia, which is injected through needle 110 once the needle has penetrated near and far walls 118, 116 of vessel 102 and again after the needle is withdrawn to a position adjacent the near wall. Similar to balloon 130, the injected solution creates spaces in the soft connective tissue adjacent near and far walls 118, 116 of vessel 102.

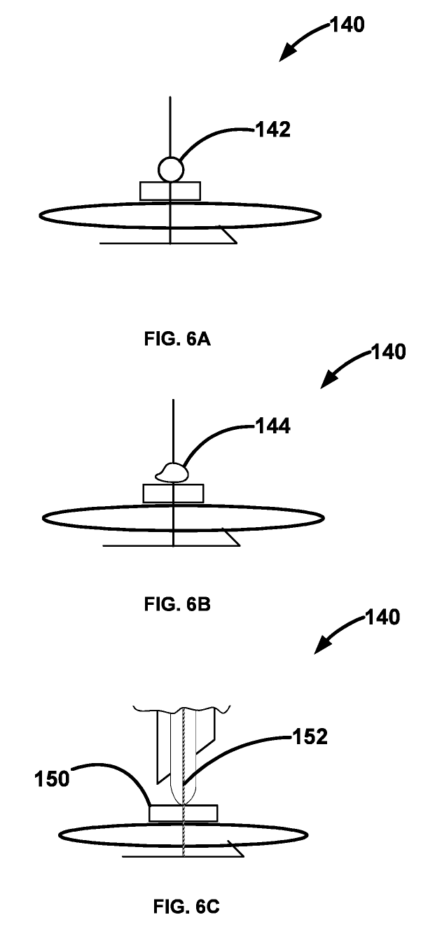

Referring now to FIGS. 6A-6C, in some embodiments, system 100 includes a locking mechanism 140 for securing near wall buttress 104 to suture ligator 114 at a position proximate to but not joined with far wall buttress 106 thereby ligating vessel 102. As shown in FIG. 6A, in some embodiments, locking mechanism 140 is formed from a knot 142, e.g., a fisherman's knot that is slipped down along suture ligator 114 to compress vessel 102 between near wall buttress 104 and far wall buttress 106 to achieve vessel ligation. Knot 142 can be formed from either a permanent or an absorbable, monofilament suture.

Referring now to FIG. 6B, in some embodiments, locking mechanism 140 is formed from an adhesive 144, e.g., a rapidly acting glue or acrylic, to secure near wall buttress 104 to suture ligator 114 at a desired position. Adhesive 144 is extruded around tip 132 of needle 110 at an interface 146 between suture ligator 114 and near wall buttress 104. In some embodiments, all materials that are implanted adjacent vessel 102, e.g., adhesive 144, etc., are either permanent or bioabsorbable.

Referring now to FIG. 6C, in some embodiments, locking mechanism 140 includes a threaded disk or bar 150 and threaded wire ligator 152. Threaded disk or bar 150 serves as near wall buttress 104 and is positioned by using plunger 154 to screw it down along ligator 152 until vessel 102 is compressed against far wall buttress 106. Threaded wire ligator 152 is then cut at a position opposite far wall buttress 106 and above threaded disk or bar 150.

Referring now to FIGS. 4 and 7A-7E, as mentioned above, in some embodiments, far wall buttress 106 is formed from a collapsible accordion portion 126. In some embodiments, collapsible accordion portion 126 is formed from a strip of biologic or bioabsorbable material. Collapsible accordion portion 126 is threaded onto suture ligator 114 like a “ribbon threaded onto a string” such that when the suture is pulled up, the collapsible accordion portion catches on far wall 116 of vessel 102, and collapses from a strip to a mushroom or flowered panel of material. This expands the surface area of portion 126 thereby securing it outside of far wall 116. In some embodiments, near wall buttress 104 is similarly formed from a second collapsible accordion portion 160. Using a plunger 162 along an axis 164 of suture ligator 114, the two flowered panels formed from collapsed accordion portions 126 and 160, under tension, collapse upon and ligate vessel 102. In some embodiments, a locking mechanism, e.g., mechanism 140 as described above, is used to hold collapsed accordion portions 126 and 160 in place on suture ligator 114. As permanent implants, biologic accordion portions 126 and 160 are reabsorbed by the body or become scar tissue avoiding the risk of infection associated with permanent, non-biologic implants.

Referring again to FIG. 3, in some embodiments, near and far wall buttresses 104 and 106 are formed from a star-shaped member 124 having radially extending members 125. Radially extending members 125 are linearly retractable along suture ligator 114. Members 125 collapse linearly to deploy and remove but expand radially when unconstrained. A temporary plunger (not shown) is used along the suture axis to secure near and far wall buttresses 104 and 106 formed from star-shaped member 124, e.g., two radial “plates,” together around the vessel to achieve temporary ligation. The plunger is then removed once the vascular manipulation or infusion has been completed. This releases the tension on near and far wall buttresses 104 and 106, and then they are be retracted into the delivery system or left in place as inert implants. Buttresses according to member 124 can be utilized for temporary vessel ligation to allow safe manipulation of or infusion into a vessel. This approach allows for temporary interruption of the vascular system by isolating the vessel being treated or manipulated. The interruption allows one to reversibly and functionally disconnect the vessel from the rest of the vascular system for as long a time as is needed to effect the goal of the manipulation and prevent an adverse outcome such as intravascular embolization or extension of infusion of a sclerosant or chemotherapeutic agent to parts of the vascular bed for which it was not intended or desired. Once the additional vascular manipulation has been performed and successfully completed, the need for the security or integrity of the ligation of the vessel becomes obsolete allowing release and or removal of buttresses 104 and 106.

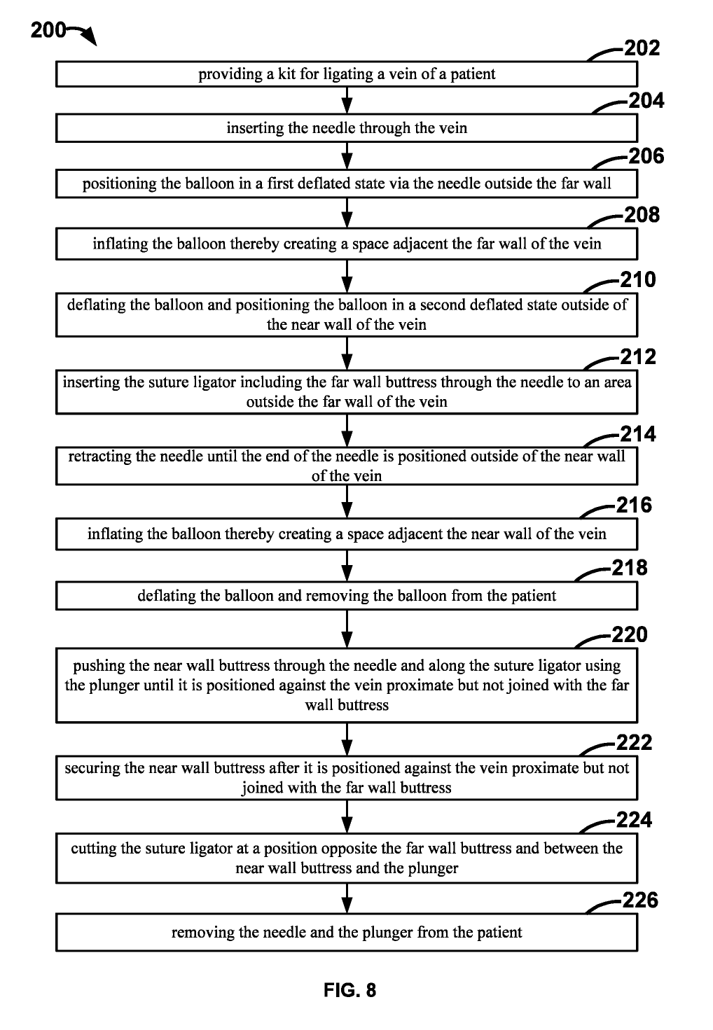

Referring now to FIG. 8, some embodiments of the disclosed subject matter include a method 200 for of ligating a vein. At 202, a kit for ligating a vein of a patient, which includes the following is provided: a delivery device including a needle; a balloon; a suture ligator including a far wall buttress, the far wall buttress being positioned outside a far wall of the vein via the needle; a near wall buttress, the near wall buttress being positioned and secured along the suture ligator outside a near wall of the vein via the needle; and a plunger for positioning the near wall buttress downwardly against the vein and securing the near wall securable buttress proximate to but not joined with the far wall buttress thereby ligating the vein, the plunger being sized to fit within the needle. At 204, the needle is inserted through the vein so that an end of the needle is positioned outside the far wall of the vein. At 206, the balloon is positioned in a first deflated state via the needle outside the far wall of the vein. At 208, the balloon is inflated thereby creating a space adjacent the far wall of the vein. At 210, the balloon is deflated and positioned in a second deflated state outside of the near wall of the vein. At 212, the suture ligator including the far wall buttress is inserted through the needle to an area outside the far wall of the vein. At 214, the needle is retracted until the end of the needle is positioned outside of the near wall of the vein. At 216, the balloon is inflated thereby creating a space adjacent the near wall of the vein. At 218, the balloon is deflated and removed from the patient. At 220, the near wall buttress is pushed through the needle and along the suture ligator using the plunger until it is positioned against the vein proximate but not joined with the far wall buttress. At 222, the near wall buttress is secured after it is positioned against the vein proximate but not joined with the far wall buttress. At 224, the suture ligator is cut at a position opposite the far wall buttress and between the near wall buttress and the plunger. At 226, the needle and plunger are removed from the patient.

In addition to the embodiments disclosed above, the following various alternative embodiments are contemplated: a) percutaneous delivery of a single ligature or multiple ligatures; b) reusable or a one-time use embodiments; c) percutaneous delivery of either a bioabsorbable ligature or a permanent ligature; d) crossing of the vessel with a needle or with a needle/wire system to allow deployment of the ligature on the near and far walls of the vessel; and e) ligation and infusion of vessels with chemical sclerosants or glues to ablate branches and contiguous vessels attached to the vessel ligated. In addition to percutaneous delivery, some embodiments include non-percutaneous delivery of a ligator, e.g., laprascopically or endoscopically.

Aspects of the disclosed subject matter provide percutaneous, minimally invasive interruption of blood vessels, which thereby avoids incisions, pain, and minimizes time of healing. Methods, systems, and apparatus according to the disclosed subject matter provide treatment of tortuous, hard to access blood vessels that are not currently easily treated by low risk minimally invasive catheter ablation techniques. Embodiments according to the disclosed subject matter provide controlled interruption of vessels without the risk of the device, or agent embolizing or effecting vessels remote to the site of treatment. Embolization techniques with chemicals, glues, sclerosants, coils or cuffs can lead to agents migrating to or perfusing vessels not targeted for interruption or ablation. This can lead to complications such as arterial ischemia, stroke, transient ischemia attack, intracardiac thrombus, deep vein thrombosis, phlebitis, and pulmonary embolism.

Although the disclosed subject matter has been described and illustrated with respect to embodiments thereof, it should be understood by those skilled in the art that features of the disclosed embodiments can be combined, rearranged, etc., to produce additional embodiments within the scope of the invention, and that various other changes, omissions, and additions may be made therein and thereto, without parting from the spirit and scope of the present invention.

What is claimed is:

1. A system for ligating a vein, comprising:
a delivery device including a needle;
a suture ligator including a far wall buttress, said far wall buttress being positioned outside a far wall of the vein via said needle;
a near wall buttress, said near wall buttress being positioned and secured along said suture ligator outside a near wall of the vein via said needle; and
a plunger for positioning said near wall buttress downwardly against the vein and securing said near wall buttress proximate to but not joined with said far wall buttress thereby ligating the vein, said plunger being sized to fit within said needle.

2. The system according to claim 1, further comprising a space creating mechanism for creating space at least one of 1) outside said near wall of the vein and 2) outside said far wall of the vein.

3. The system according to claim 2, wherein said space creating mechanism includes a balloon.

4. The system according to claim 2, wherein said space creating mechanism includes a solution.

5. The system according to claim 4, wherein said solution is saline or tumescent anesthesia.

6. The system according to claim 1, wherein at least one of said near and far wall buttresses includes a barbed shoe.

7. The system according to claim 1, wherein at least one of said far and near wall buttresses include a collapsible accordion portion.

8. The system according to claim 7, wherein said collapsible accordion portion is formed from a biologic or bioabsorbable material.

9. The system according to claim 1, wherein at least one of said far and near wall buttresses include radially extending members.

10. The system according to claim 9, wherein said radially extending members are linearly retractable along said suture ligator.

11. The system according to claim 1, wherein said near wall buttress includes a knot for securing said near wall buttress to said suture ligator.

12. The system according to claim 1, wherein said near wall buttress includes a threaded disk and said suture ligator is threaded.

13. The system according to claim 1, wherein said near wall buttress includes an adhesive for securing said near wall buttress to said suture ligator.

14. The system according to claim 3, further comprising a guidewire and catheter positioned in said delivery device for facilitating placement of said near and far wall buttresses.

15. A system for ligating a blood vessel, comprising:
a delivery device including a needle;
a space creating mechanism for creating space at least one of 1) outside a near wall of the vessel and 2) outside a far wall of the vessel;
a suture ligator including a far wall buttress, said far wall buttress being positioned outside said far wall of the vessel via said needle;
a near wall buttress, said near wall buttress being positioned and secured along said suture ligator outside said near wall of the vessel via said needle;
a plunger for positioning said near wall buttress downwardly at a position along said suture ligator so that said near wall buttress compresses the vessel but is not joined with said far wall buttress, said plunger being sized to fit within said needle; and
a locking mechanism for securing said near wall buttress to said suture ligator at a position proximate to but not joined with said far wall buttress thereby ligating the vessel.

16. The system according to claim 15, wherein said space creating mechanism includes one of a balloon or a injectable fluid.

17. The system according to claim 15, wherein said locking mechanism includes at least one of a knot, a disk or bar, and an adhesive, or a screw down near wall buttress.

18. A method of ligating a vein, said method comprising:
providing a kit for ligating a vein of a patient including a delivery device including a needle, a suture ligator including a far wall buttress, said far wall buttress being positioned outside a far wall of the vein via said needle, a near wall buttress, said near wall buttress being positioned and secured along said suture ligator outside a near wall of the vein via said needle, and a plunger for positioning said near wall buttress downwardly against the vein and securing said near wall securable buttress proximate to but not joined with said far wall buttress thereby ligating the vein, said plunger being sized to fit within said needle;
inserting said needle through the vein so that an end of said needle is positioned outside said far wall of the vein;
inserting said suture ligator including said far wall buttress through said needle to an area outside said far wall of the vein;
retracting said needle until said end of said needle is positioned outside of said near wall of the vein;
pushing said near wall buttress through said needle and along said suture ligator using said plunger until it is positioned against the vein proximate but not joined with said far wall buttress;
securing said near wall buttress after it is positioned against the vein proximate but not joined with said far wall buttress;
cutting said suture ligator at a position opposite said far wall buttress and between said near wall buttress and said plunger; and
removing said needle and said plunger from the patient.

19. The method according to claim 18, wherein said kit further comprises a balloon.

20. The method according to claim 19, further comprising:
positioning said balloon in a first deflated state via said needle outside said far wall of the vein;
inflating said balloon thereby creating a space adjacent said far wall of the vein;
deflating said balloon and positioning said balloon in a second deflated state outside of said near wall of the vein;
inflating said balloon thereby creating a space adjacent said near wall of the vein; and
deflating said balloon and removing said balloon from the patient.
